(19) United States
(12) Patent Application Publication
(10) Pub. No.: US 2023/0005586 A1
(43) Pub. Date: Jan. 5, 2023

(54) DETERMINING TOTAL DAILY BASAL DOSE MISMATCH

(71) Applicant: Bigfoot Biomedical, Inc., Milpitas, CA (US)

(72) Inventor: Zahra Eghtesadi, Milpitas, CA (US)

(21) Appl. No.: 17/756,585

(22) PCT Filed: Nov. 30, 2020
(86) PCT No.: PCT/US2020/070828

§ 371 Date: May 27, 2022

Related U.S. Application Data
(60) Provisional application No. 62/943,150, filed on Dec. 3, 2019.

(51) Int. Cl. G16H 20/17; G16H 50/20

(52) U.S. Cl. CPC G16H 20/17; G16H 50/20

(57) ABSTRACT

A method comprises: receiving, in a computer system, data regarding insulin therapy treatment of a person with diabetes, the data relating to a time period and comprising blood glucose values for the person with diabetes and time-of-administration information for the insulin therapy treatment; determining, using the computer system and based on the blood glucose values and the time-of-administration information, a total daily basal dose (TDBD) mismatch value for the person with diabetes; and generating, using the computer system, an output based on the determined TDBD mismatch value.

### CROSS-REFERENCE TO RELATED APPLICATIONS

This application claims priority to U.S. Provisional Patent Application No. 62/943,150, entitled “DETERMINING TOTAL DAILY BASAL DOSE MISMATCH”, the disclosure of which is incorporated by reference herein in its entirety.

### TECHNICAL FIELD

This document relates to determining total daily basal dose mismatch.

### BACKGROUND

Diabetes mellitus is a chronic metabolic disorder caused by the inability of a person's pancreas to produce sufficient amounts of the hormone insulin such that the person's metabolism is unable to provide for the proper absorption of sugar and starch. This failure leads to hyperglycemia, i.e., the presence of an excessive amount of glucose within the blood plasma. Persistent hyperglycemia has been associated with a variety of serious symptoms and life threatening long-term complications such as dehydration, ketoacidosis, diabetic coma, cardiovascular diseases, chronic renal failure, retinal damage and nerve damages with the risk of amputation of extremities. Because healing is not yet possible, a permanent therapy is necessary which provides constant glycemic control in order to constantly maintain the level of blood analyte within normal limits. Such glycemic control is achieved by regularly supplying external drugs to the body of the patient to thereby reduce the elevated levels of blood analyte. An external biologically effective drug (e.g., insulin or its analog) is commonly administered by means of daily injections. In some cases, multiple, daily injections of a mixture of rapid- and long-acting insulin are administered via a reusable transdermal liquid dosing device.

### SUMMARY

In a first aspect, a method comprises: receiving, in a computer system, data regarding insulin therapy treatment of a person with diabetes, the data relating to a time period and comprising blood glucose values for the person with diabetes and time-of-administration information for the insulin therapy treatment; determining, using the computer system and based on the blood glucose values and the time-of-administration information, a total daily basal dose (TDBD) mismatch value for the person with diabetes; and generating, using the computer system, an output based on the determined TDBD mismatch value.

Implementations can include any or all of the following features. Determining the TDBD mismatch value comprises using a regression model. The method further comprises training the regression model before determining the TDBD mismatch value, the regression model trained using simulated insulin therapy treatment data. Generating the output comprises adjusting an insulin therapy setting for the person with diabetes using the TDBD mismatch value. The insulin therapy setting is adjusted by an entirety of the TDBD mismatch value. The insulin therapy setting is adjusted by a portion of the TDBD mismatch value. The method further comprises communicating the adjusted insulin therapy setting using the computer system. The adjusted insulin therapy setting is communicated to at least one of the person with diabetes or a physician for the person with diabetes. Generating the output comprises generating a communication. The communication is generated to at least one of the person with diabetes or a physician for the person with diabetes. The method further comprises determining, using the computer system and based on the TDBD mismatch value, an insulin sensitivity factor (ISF) for the person with diabetes. The ISF is determined using a trivariate relationship. The ISF is determined based on a new insulin therapy value calculated using the TDBD mismatch value. The method further comprises determining, using the computer system and based on the TDBD mismatch value, a carbohydrate-to-insulin ratio (CR) for the person with diabetes. The CR is determined using a trivariate relationship. The CR is determined based on a new insulin therapy value calculated using the TDBD mismatch value. A programmed TDBD value for the person with diabetes for the time period is known, and wherein generating the output comprises adjusting the programmed TDBD value using the TDBD mismatch value. Adjusting the programmed TDBD value comprises determining a new TDBD value for the person with diabetes by dividing the programmed TDBD value with the TDBD mismatch value. The time period is two weeks.

In a second aspect, a method comprises: obtaining, in a computer system, data regarding insulin-based management of diabetes, the data including blood glucose values and time-of-administration information for the insulin-based management of diabetes; training, using the computer system, a regression model using the data, the regression model trained to determine a total daily basal dose (TDBD) mismatch value; and obtaining, using the computer system, a TDBD mismatch value using the trained regression model.

Implementations can include any or all of the following features. The method further comprises: training multiple candidate regression models using the data; and selecting one of the multiple candidate regression models to be the regression model, the selection comprising: determining a predictive ability for each of the multiple candidate regression models; and identifying one of the multiple candidate regression models having a highest predictive ability of the determined predictive abilities. The method further comprises: constructing feature sets, wherein each of the feature sets is constructed by selecting one or more features to include in the feature set; and performing a feature selection process using the feature sets and the data to obtain a training feature set. The training feature set includes at least some of: a feature reflecting time below a first blood glucose value; a feature reflecting time below a second blood glucose value; a feature reflecting time with blood glucose within a predefined range; a feature reflecting time above a third blood glucose value; a feature reflecting time above a fourth blood glucose value; a feature reflecting a geometric mean of blood glucose values; a feature reflecting a geometric standard deviation of the blood glucose values; a feature reflecting a loss function regarding the blood glucose values; a feature reflecting a programmed TDBD value; a feature reflecting a programmed carbohydrate-to-insulin ratio; or a feature reflecting a programmed insulin sensitivity factor. The method further comprises: simulating insulin-based management of diabetes; and obtaining the data regarding insulin-based management of diabetes from simulating the insulin-based management of diabetes.

In a third aspect, a system comprises: a non-transitory storage medium having stored therein: data regarding insulin therapy treatment of a person with diabetes, the data relating to a time period and comprising blood glucose values for the person with diabetes and time-of-administration information for the insulin therapy treatment; and a mismatch regression model; and at least one processor to provide the data to the mismatch regression model and determine a total daily basal dose (TDBD) mismatch value for the person with diabetes.

Implementations can include any or all of the following features. The system further comprises an output component to generate an output based on the determined TDBD mismatch value. The output comprises an adjustment of an insulin therapy setting for the person with diabetes using the TDBD mismatch value. The output comprises a communication.

### BRIEF DESCRIPTION OF DRAWINGS

FIG. 1 shows an example of a system for training a total daily basal dose (TDBD) regression model.

FIG. 2 shows a simplified block diagram of a computing platform for determining a TDBD mismatch, in accordance with one or more embodiments.

FIG. 3 shows a graph of an example of time in range according to a prior approach.

FIG. 4 shows a graph of an example of a geometric mean for a full population of 100 subjects according to a prior approach.

FIG. 5 shows a graph of an example of time in range according to the present subject matter.

FIG. 6 shows a graph of an example of a geometric mean for a full population according to the present subject matter.

FIG. 7 shows a graph of an example of a geometric mean for a full population of 300 subjects according to the present subject matter.

FIG. 8 shows a simplified block diagram of an example of a system for insulin-based management of diabetes.

FIG. 9 shows a functional block diagram of a system for training a TDBD regression model using machine learning, in accordance with one or more embodiments.

FIG. 10 shows an example of selecting time periods for evaluating blood glucose data.

FIG. 11 shows an example of a computer device that can be used to implement the techniques described here.

Like reference symbols in the various drawings indicate like elements.

### DETAILED DESCRIPTION

This document describes examples of systems and techniques for improving a convergence of insulin therapy. In some implementations, a mismatch value regarding total daily basal dose (TDBD) can be determined for a person with diabetes and used for one or more purposes. For example, the TDBD mismatch value can be determined using a regression model that has been trained based on data for a population (e.g., actual data or simulated data).

Insulin delivery devices include, but are not limited to, insulin injection pens, insulin inhalers, insulin pumps, and insulin syringes. The improper dosing of insulin, whether due to human error, malfunction of an insulin pen, skipping doses, double dosing, or incorrect dosing, is always a concern. Methods, devices, and systems provided herein are described for the delivery of insulin, collection of blood glucose data, and/or the treatment of diabetes. Moreover, methods, devices, and systems provided herein may be adapted for the delivery of other medications, the collection of other analyte data, and/or the treatment of other diseases. Methods, devices, and systems provided herein are described by exemplifying features and functionalities of a number of illustrative embodiments. Other implementations are also possible.

Some examples herein refer to an insulin injection pen. An insulin injection pen includes at least one container holding insulin (e.g., an insulin cartridge), a dial or other mechanism to specify a dose, and a pen needle for transcutaneous delivery of the insulin into the tissue or vasculature of the person with diabetes. In a reusable insulin injection pen, the insulin container (e.g., the cartridge) is replaceable or refillable. A prefilled insulin injection pen is intended for use during a limited time. The dose-specifying mechanism can include a rotatable wheel coupled to mechanics and/or electronics for capping the administered amount of insulin at the volume of the specified dose (e.g., in terms of a number of units of insulin). The dose-specifying mechanism can have a mechanical and/or electronic display that reflects the current setting of the mechanism. The pen needle can be permanently attached to the housing of the insulin injection pen (e.g., as in the case with a disposable pen), or it can be removable (e.g., so that a new needle can be applied when needed). For example, a replaceable pen needle can include a hollow needle affixed to a fitting configured for removeable attachment to an end of the insulin injection pen.

Some examples herein refer to a mobile communication device. As used herein a mobile communication device includes, but is not limited to, a mobile phone, a smartphone, a wearable electronic device (e.g., a smart watch), a tablet, a laptop computer, a portable computer, and similar devices. A mobile communication device includes one or more processors, a non-transitory storage device (e.g., a memory and/or a hard drive) holding executable instructions for operating the mobile communication device, wireless communication components, and one or more input and/or output devices (e.g., a touchscreen, display, or keyboard). The mobile communication device can operate according to one or more application programs stored locally on the mobile communication device, or remotely (e.g., when using cloud computing), or combinations thereof. The mobile communication device can execute at least one operating system in order to perform functions and services.

Some examples herein refer to a continuous glucose monitor (CGM). A CGM is an electronic device configured to take readings of glucose values on an ongoing basis or at regular intervals in order to estimate the blood glucose level of the person with diabetes. Some CGMs determine blood glucose values periodically (e.g., after a certain number of seconds or minutes) and output the information automatically or upon being prompted. The CGM may include wireless communication components for one or more types of signaling, including, but not limited to, Near-Field Communication (NFC) and/or Bluetooth communication.

In some embodiments, systems, devices, and methods provided herein can recommend insulin doses (e.g., dosages of long-acting and/or rapid-acting insulin) using any suitable technique. In some embodiments, recommended insulin dosages may be based upon blood glucose data (e.g., current estimated glucose value (EGV) from a CGM), flash glucose monitor, blood glucose meter, or any other sensor, blood glucose trend data, etc.), insulin administration data (bolus dosage amounts of rapid-acting insulin, dosages of long-acting insulin, dosage times, calculation of Insulin-on-Board (“IOB”) and/or active insulin, etc.), meal data (mealtimes, user estimated carbohydrates, user estimated meal categorizations, user estimated glycemic impact of meal user meal history, user meal trends, etc.), and/or one or more insulin deliver parameters: total daily dose of basal insulin or long-acting insulin, carbohydrate-to-insulin ratio (CR), insulin sensitivity factor (ISF), etc.). Methods, devices and systems provided herein can, in some embodiments, adjust insulin delivery parameters over time based on glucose data and/or insulin administration data.

Some examples herein refer to long-acting insulin and rapid-acting insulin, or in some cases more generally to first and second types of insulin. Insulin used for therapeutic treatment is often synthesized human insulin. Moreover, different insulins can be characterized in how quickly they typically begin to work in the body of the person with diabetes after administration, and/or how long they typically remain active in the body of the person with diabetes. Rapid-acting insulin can be used to dose for meals or to correct high blood sugar. There is more than one type of insulin that can be considered a rapid-acting insulin. Many, but not necessary all, rapid-acting insulins begin working within about one hour after administration. Similarly, there is more than one type of insulin that can be considered a long-acting insulin, and many, but not necessarily all, long-acting insulins begin working about one hour or longer after administration. Long-acting insulin is often referred to as basal insulin (e.g., insulin used to support basic metabolic needs). Generally, a long-acting insulin has a greater active time (i.e., the length of time that the insulin continues to be active in the body of the person with diabetes after administration) than a rapid-acting insulin. As such, a long-acting insulin is an example of a type of insulin having an active time that is longer than an active time for a type of insulin such as a rapid-acting insulin.

Examples herein refer to TDBD. The TDBD can reflect a setting of an insulin therapy corresponding to the amount of long-acting insulin to be administered. The actual TDBD of a person with diabetes may not be known. Rather, a programmed TDBD value can be entered into a diabetes therapy management system for the person and can be adjusted as needed. A TDBD mismatch value, moreover, can represent a determination of a relationship between the programmed TDBD value and the unknown actual TDBD value. In some implementations, the determined TDBD mismatch value represents a division of the programmed TDBD value with the unknown actual TDBD value. For example, a new (i.e., adjusted) TDBD value can be calculated by dividing the programmed TDBD value by the determined TDBD mismatch value. The term TDBD is sometimes often applied in the context of insulin therapy administration by way of a pump device. Other terms can be used to refer to the dosage of long-acting insulin over a 24-hour period, including, but not limited to, daily long-acting insulin. As used herein, the term TDBD also includes the concept of daily long-acting insulin.

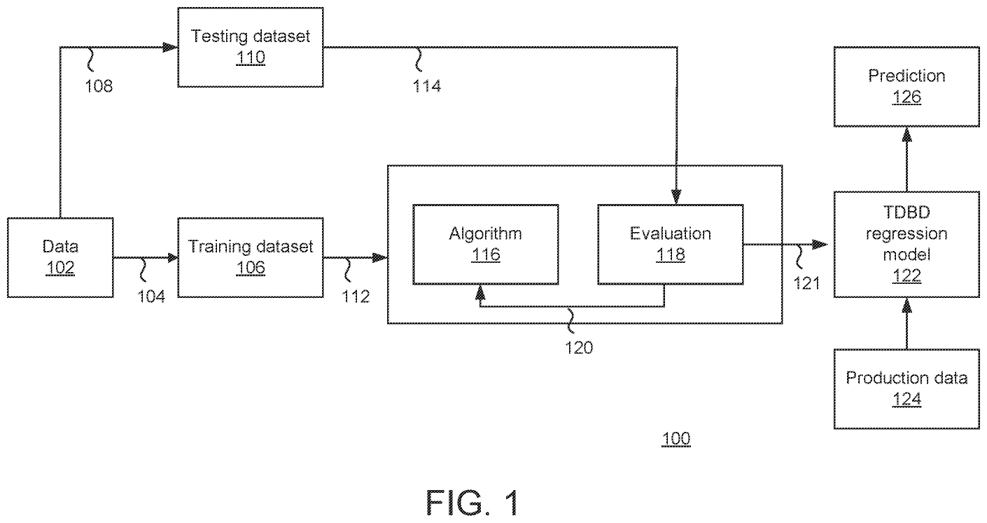

FIG. 1 shows an example of a system 100 for training a TDBD regression model. The system 100 can be used with one or more other examples described elsewhere herein. The system 100 can involve the following stages: gathering data 102; preprocessing the data 102 at 104 (into a training dataset 106) and at 108 (into a testing dataset 110); researching the regression model, in terms of an algorithm 116 and an evaluation 118 that are based on the training dataset 106 at 112, by way of one or more iterations 120; testing the regression model using the testing dataset 110 at 114; training, at 121, a TDBD regression model 122; and evaluating the TDBD regression model 122 using production data 124 to make one or more predictions 126. In some implementations, the training dataset 106 can include both a set of training data and a set of validation data.

The data 102 can include actual patient data or simulated data, or combinations thereof. For example, data can be provided on a real-time basis or from storage. The data 102 can be preprocessed to clean the data 102. For example, the data 102 can be converted from a raw format (e.g., a format as collected) into a format suitable for regression analysis. As another example, noise can be removed from the data 102, and/or inconsistencies in the data 102 can be removed. As another example, missing values in the data 102 can be ignored (e.g., by removing a remainder of a column or row of the missing value). The training can involve supervised or unsupervised learning. In some implementations, supervised learning involves regression to learn how to predict a numerical value of a continuous variable.

The TDBD regression model 122 can take as inputs at least blood glucose values for a person with diabetes and time-of-administration information for the insulin therapy treatment. In some implementations, the blood glucose values are read, estimated, or inferred using one or more devices (e.g., a CGM or a BGM). For example, blood glucose values can be collected from the person with diabetes at regular intervals. In some implementations, the time-of-administration information (e.g., when the person ingested insulin, sometimes referred to as time-of-bolus information) can be read, estimated, or inferred using one or more devices (e.g., an insulin injection pen, or a pen cap of the insulin injection pen). For example, time-of-administration information can be collected from the person with diabetes at regular intervals.

In some implementations, the diabetes therapy management system (e.g., a smartphone, insulin injection pen, or a pen cap) facilitates receipt of dose times data (e.g., by way of detecting capping data from a pen cap of an insulin injection pen, or using another proxy for dosage times). In some implementations, pen cap usage data can be detected. For example, the pen cap usage data can be based on one or more capping or uncapping events of a pen cap for an insulin injection pen. One or more of dose times data or pen cap usage data can be used in some implementations. For example, the dose times data can be determined or inferred from the pen cap usage data. In some implementations, about two weeks of blood glucose data and dosage timing information can be received. In some implementations, the time period between therapy parameter adjustments can be configurable by an algorithm. For example, the time period between consecutive therapy parameter adjustments can be referred to as an iteration of the algorithm.

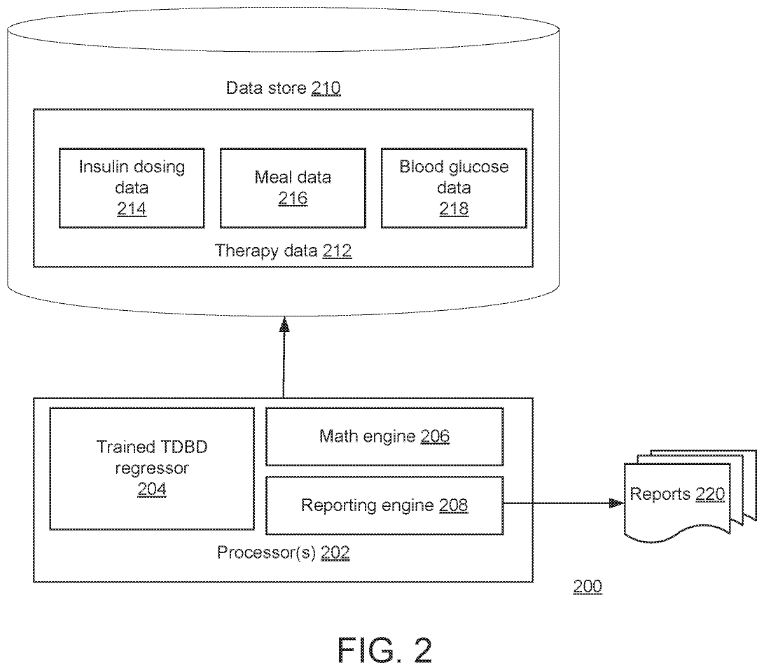

FIG. 2 shows a computing platform 200 for determining a TDBD mismatch value, in accordance with disclosed embodiments. The computing platform 200 can be used with one or more other examples described elsewhere herein. In disclosed embodiments, computing platform 200 is, or is operative to be executed as, a data processing system, and more specifically, as a data processing system for performing TDBD mismatch determination. Computing platform 200 may include data store 210 and processor(s) 202. Data store 210 may include therapy data 212. Therapy data 212 is data related to insulin therapy of one or more persons. Therapy data 212 can include insulin dosing data 214, meal data 216, and blood glucose data 218. Alternatively, or additionally, therapy data 212 may include exercise data, sleep data, and/or physiological parameters of a patient (e.g., an insulin sensitivity factor or insulin-to-carbohydrate ratio).

Blood glucose data 218 may include data about blood glucose in a human body at one or more times. Blood glucose data 218 may include measurements of blood glucose levels, for example, raw blood glucose measurements, blood glucose estimates based on blood glucose measurements, and/or aggregations of the same (e.g., averages, trends and metrics). Blood glucose data 218 may include date and time (e.g., a timestamp), and a value for each blood glucose measurement. In disclosed embodiments, any suitable glucose sensor may provide blood glucose data 218, for example, a continuous glucose monitor (CGM), a flash glucose monitor, a blood glucose meter (BGM). In the case of CGMs and flash glucose monitors, they may be configured to provide blood glucose data 218 based on interstitial fluid glucose levels of a person, which may be correlated to blood glucose levels. A BGM may be configured to provide blood glucose data based on a blood sample. Accordingly, the term “blood glucose” is not limited herein to using just blood glucose data, values, levels etc., but is also intended to include interstitial fluid glucose levels, intermediate measurements, and legal equivalents thereof.

Insulin dosing data 214 may include dosing event data. Dosing event data may include data about insulin dosing actions at one or more times and may include, for example, a dosing time or time range, type of insulin (e.g., LA insulin and rapid acting (RA) insulin) dosed, brand of insulin, and/or amount of dosed insulin. In some embodiments, dosing event data may include an indication of a dosing mechanism, for example, injection pen, inhaler, or infusion pump. In some embodiments, dosing event data may include an indication of whether dosing event data, in part or in whole, is based on an actual dosing action (e.g., detecting insulin delivery, for example, based on a manual action of a pump or a control signal configured to cause insulin delivery), user tracking of dosing actions (e.g., a PWD or caregiver enters a dose using a therapy application executing on a mobile device), or inferred dosing actions (e.g., from capping/uncapping of an injection pen).

Processor(s) 202 may be configured to execute a number of engines for performing disclosed embodiments. Processor(s) 202 can include a trained TDBD regressor 204, math engine 206, and reporting engine 208.

Trained TDBD regressor 204 may be configured, generally, to process therapy data 212 or part(s) of therapy data 212, and determine one or more TDBD mismatches. A part of therapy data 212 processed by the trained TDBD regressor 204 may correspond to a particular time period. In one embodiment, Trained TDBD regressor 204 may return predictions of a continuous variable, such as to determine a TDBD mismatch. Trained TDBD regressor 204 may output a determined TDBD mismatch, or can cause one or more other actions to be taken. Trained TDBD regressor 204 may be trained using one or more supervised and/or unsupervised learning techniques, including those described in more detail in this disclosure.

Math engine 206 may be configured to perform various statistical calculations using therapy data 212 and results provided by trained TDBD regressor 204. In various embodiments, statistical calculations may include, for example, frequency calculations, confidence calculations, probability calculations, and more.

Reporting engine 208 may be configured, generally, to generate one or more reports 220 responsive to trained TDBD regressor 204 and/or math engine 206. Reports 220 may include descriptions of retrospective studies performed at computing platform 200 as more fully described elsewhere herein, and may include, for example, patient identifiers, descriptions of retrospective time periods, assigned class labels, the class labels and more.

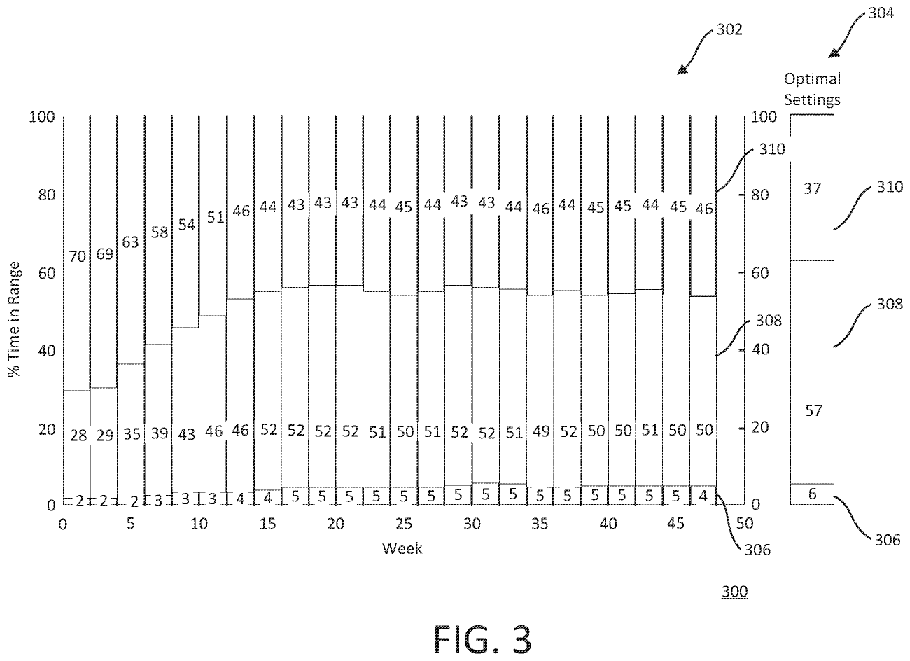

FIG. 3 shows a graph 300 of an example of time in range according to a prior approach. The graph 300 includes data 302 and optimal data 304. Each of the data 302 and optimal data 304 shows percentages of an overall population. In some implementations, the data 302 can represent an actual population or a simulated population. For example, the graph 300 can represent a simulation of 100 subjects for 24 iterations totaling 366 days. The optimal data 304 corresponds to an ideal distribution of blood glucose values for an optimal insulin therapy management.

The data 302 shows time along the horizontal axis (e.g., in terms of weeks), and the respective percentages of time spent in different blood glucose ranges along the vertical axis. In some implementations, a range 306 corresponds to less than 70 mg/dl; a range 308 corresponds to 70-180 mg/dl; and a range 310 corresponds to more than 180 mg/dl. The ranges 306, 308 and 310 apply to both the data 302 and the optimal data 304. For example, in this example, at week zero in the data 302, the range 306 has two percent; the range 308 has 27 percent; and the range 310 has 70 percent. That is, at week zero the population spent two percent of its time with less than 70 mg/dl; the population spent 27 percent of its time in the range 70-180 mg/dl; and the population spent 70 percent of its time at more than 180 mg/dl. It can be seen that at week zero, the population reflected in the data 302 spent less time in the range 308 than according to the optimal data 304, and spent more time in the range 310 than according to the optimal data 304.

The situation indicated above, where the population at week zero is not in an optimal distribution of blood glucose ranges, can trigger a change in insulin therapy, for example by adjusting one or more insulin-therapy settings. In contrast to the present subject matter, the graph 300 is associated with a prior approach where the adjustments were limited to a certain amount per time period. In some implementations, the population of the graph 300 was evaluated after each iteration, which in this example corresponds to a two-week period. At the beginning of the next iteration, it was possible to take any of six different actions for each person: adjust an overnight (i.e., long-acting) dose upward or downward, adjust an after-meal (i.e., rapid-acting) dose upward or downward, or adjust a correction dose upward or downward. Because the prior approach did not have the benefit of TDBD-mismatch determination according to the present subject matter, it could not take into account, in say week two, whether the person with diabetes had a mismatch between the TDBD they required and the TDBD that was programmed into their insulin therapy management device (e.g., a smartphone, pen cap, or a CGM). Rather, the prior approach would allow only up to a 10% change in the insulin therapy parameter (e.g., the long-acting insulin) per iteration.

The prior approach had the advantage that the capped therapy changes that could take place every iteration eventually brought the population closer to the balance between the ranges 306, 308, and 310 that the optimal data 304 has. For example, at a time around week 20 in the data 302 (i.e., after ten iterations of two weeks each), the range 308 reaches a somewhat stable value (here, about 50%) and after that there are only small fluctuations that do not reflect an upward or downward trend. It can be said that the data 302 converges around week 20 in this example.

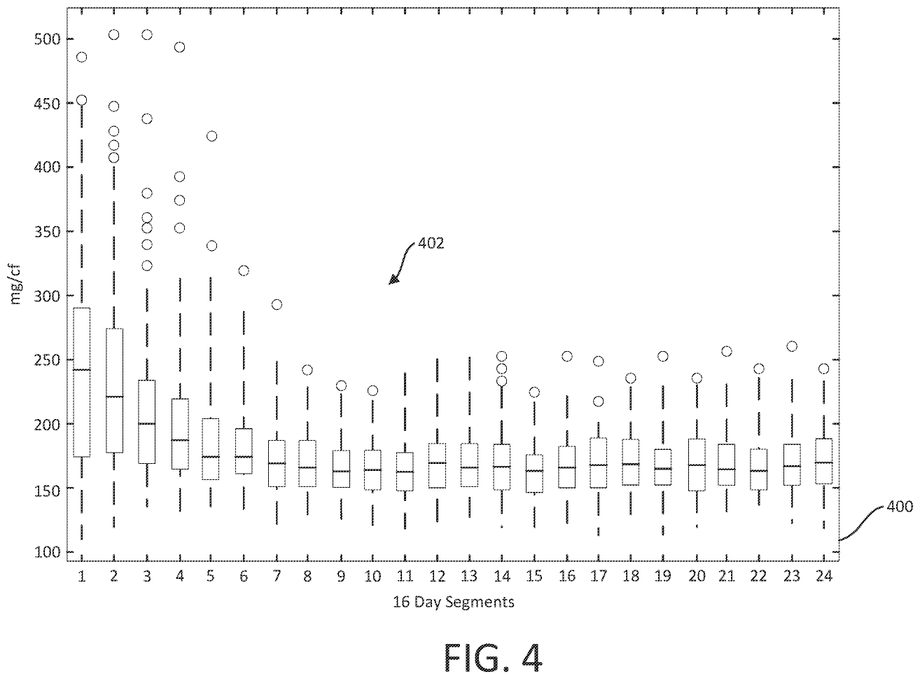

However, the prior approach reaches convergence after a significant amount of time (e.g., 20 weeks). The extensive period of time spent with a distribution of ranges that is significantly different from that of the optimal data 304 can have negative effects of the health and/or the quality of life of the population. FIG. 4 shows a graph 400 of an example of a geometric mean for a full population according to a prior approach. The graph 400 can be based on the same body of data as the graph 300 (FIG. 3) and likewise shows values 402 that exhibit a relatively slow progress toward convergence. For example, only after about ten iterations (i.e., after about 20 weeks) do the values 402 begin to stabilize as a result of the adjustments in insulin therapy that are capped each iteration and that do not have the benefit of a determination of TDBD mismatch.

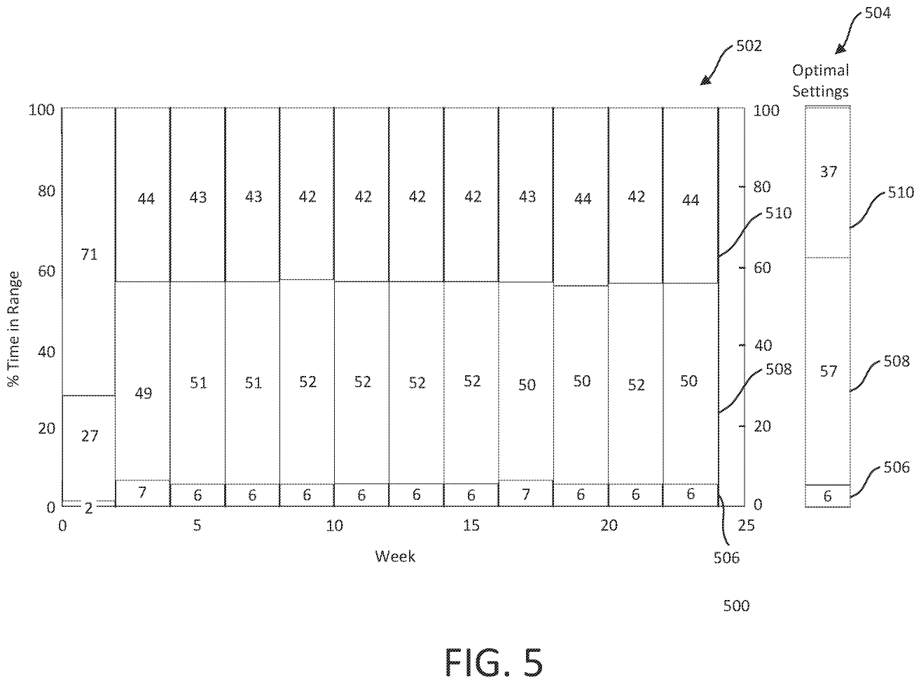

With determination of TDBD mismatch, however, insulin therapy management can be beneficially improved. For example, adjustments in insulin therapy settings can converge significantly faster than with the prior approach. FIG. 5 shows a graph 500 of an example of time in range according to the present subject matter. The graph 500 includes data 502 and optimal data 504. Each of the data 502 and optimal data 504 shows percentages of an overall population. In some implementations, the data 502 can represent an actual population or a simulated population. For example, the graph 500 can represent a simulation of 100 subjects for 24 iterations totaling 366 days. The optimal data 504 corresponds to an ideal distribution of blood glucose values for an optimal insulin therapy management.

The data 502 shows time along the horizontal axis (e.g., in terms of weeks), and the respective percentages of time spent in different blood glucose ranges along the vertical axis. In some implementations, a range 506 corresponds to less than 70 mg/dl; a range 508 corresponds to 70-180 mg/dl; and a range 510 corresponds to more than 180 mg/dl. The ranges 506, 508 and 510 apply to both the data 502 and the optimal data 504. For example, in this example, at week zero in the data 502, the range 506 has two percent; the range 508 has 26 percent; and the range 510 has 71 percent. That is, at week zero the population spent two percent of its time with less than 70 mg/dl; the population spent 26 percent of its time in the range 70-180 mg/dl; and the population spent 71 percent of its time at more than 180 mg/dl. It can be seen that at week zero, the population reflected in the data 302 spent less time in the range 508 than according to the optimal data 504, and spent more time in the range 510 than according to the optimal data 504.

The situation indicated above, where the population at week zero is not in an optimal distribution of blood glucose ranges, can trigger a change in insulin therapy, for example by adjusting one or more insulin-therapy settings. According to the present subject matter, the graph 500 is associated with an approach that takes into account a determined TDBD mismatch. In some implementations, the population of the graph 500 was evaluated after each iteration, which in this example corresponds to a two-week period. At the beginning of the next iteration, it was possible to take any of six different actions for each person: adjust an overnight (i.e., long-acting) dose upward or downward, adjust an after-meal (i.e., rapid-acting) dose upward or downward, or adjust a correction dose upward or downward. Importantly, the adjustment for the next iteration was not capped at a predefined amount but rather could be set based on the determined TDBD mismatch. In some implementations, the adjustment is set to compensate for the entire TDBD mismatch in each new iteration. That is, the insulin therapy setting can be adjusted by an entirety of the TDBD mismatch value. In some implementations, the adjustment does not compensate for the entire TDBD mismatch in each new iteration. That is, the insulin therapy setting can be adjusted by a portion of the TDBD mismatch value.

The present subject matter can have the advantage that the therapy changes that take place every iteration eventually bring the population closer to the balance between the ranges 506, 508, and 510 that the optimal data 504 has. For example, at a time around week two in the data 502 (i.e., after one iteration of two weeks), the range 508 reaches a somewhat stable value (here, about 50%) and after that there are only small fluctuations that do not reflect an upward or downward trend. It can be said that the data 502 converges around week two in this example.

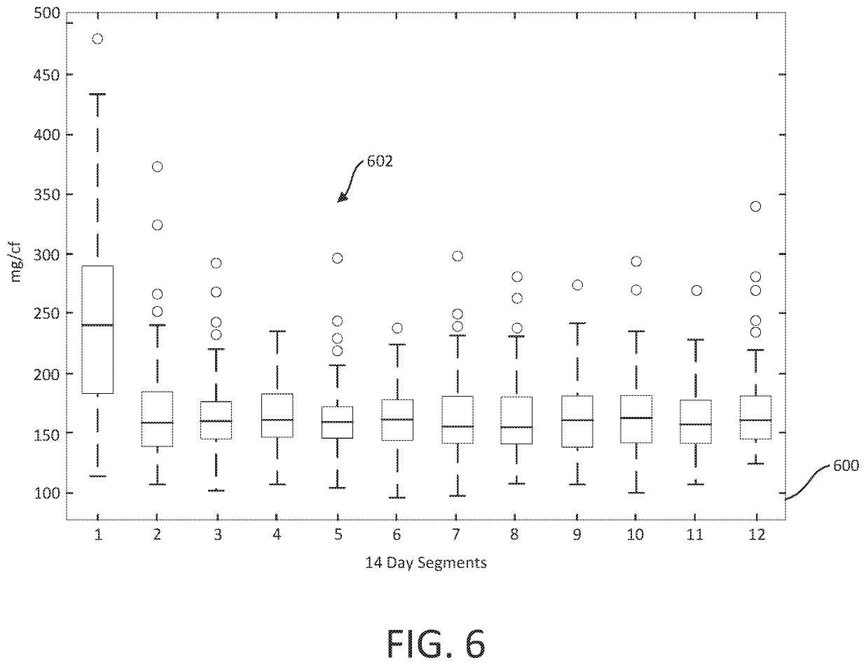

Moreover, the present subject matter can reach convergence relatively quickly. For example, after about one iteration (here, two weeks), convergence can be obtained. This can eliminate or reduce the occurrence of negative effects of the health and/or the quality of life of the population. FIG. 6 shows a graph 600 of an example of a geometric mean for a full population according to the present subject matter. The graph 600 can be based on the same body of data as the graph 500 (FIG. 5) and likewise shows values 602 that exhibit a relatively fast progress toward convergence. For example, after only about one iteration (i.e., after about two weeks) the values 602 begin to stabilize as a result of the adjustments in insulin therapy that have the benefit of a determination of TDBD mismatch.

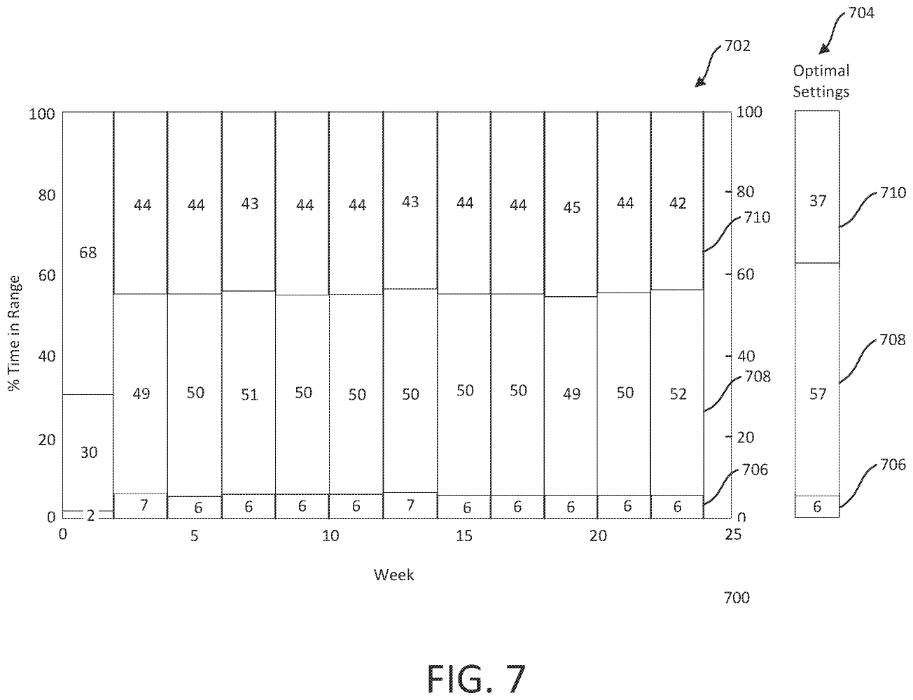

FIG. 7 shows a graph 700 of an example of a geometric mean for a full population of 300 subjects according to the present subject matter. The graph 700 includes data 702 and optimal data 704. Each of the data 702 and optimal data 704 shows percentages of an overall population. In some implementations, the data 702 can represent an actual population or a simulated population. For example, the graph 700 can represent a simulation of 300 subjects for 24 iterations totaling 366 days. The optimal data 704 corresponds to an ideal distribution of blood glucose values for an optimal insulin therapy management.

The data 702 shows time along the horizontal axis (e.g., in terms of weeks), and the respective percentages of time spent in different blood glucose ranges along the vertical axis. In some implementations, a range 706 corresponds to less than 70 mg/dl; a range 708 corresponds to 70-180 mg/dl; and a range 710 corresponds to more than 180 mg/dl. The ranges 706, 708 and 710 apply to both the data 702 and the optimal data 704. For example, in this example, at week zero in the data 702, the range 706 has two percent; the range 708 has 28 percent; and the range 710 has 68 percent. That is, at week zero the population spent two percent of its time with less than 70 mg/dl; the population spent 28 percent of its time in the range 70-180 mg/dl; and the population spent 68 percent of its time at more than 180 mg/dl. It can be seen that at week zero, the population reflected in the data 702 spent less time in the range 708 than according to the optimal data 704, and spent more time in the range 710 than according to the optimal data 704.

The situation indicated above, where the population at week zero is not in an optimal distribution of blood glucose ranges, can trigger a change in insulin therapy, for example by adjusting one or more insulin-therapy settings. According to the present subject matter, the graph 700 is associated with an approach that takes into account a determined TDBD mismatch. In some implementations, the population of the graph 700 was evaluated after each iteration, which in this example corresponds to a two-week period. At the beginning of the next iteration, it was possible to take any of six different actions for each person: adjust an overnight (i.e., long-acting) dose upward or downward, adjust an after-meal (i.e., rapid-acting) dose upward or downward, or adjust a correction dose upward or downward. Importantly, the adjustment for the next iteration was not capped at a predefined amount but rather could be set based on the determined TDBD mismatch. In some implementations, the adjustment is set to compensate for the entire TDBD mismatch in each new iteration. That is, the insulin therapy setting can be adjusted by an entirety of the TDBD mismatch value. In some implementations, the adjustment does not compensate for the entire TDBD mismatch in each new iteration. That is, the insulin therapy setting can be adjusted by a portion of the TDBD mismatch value.

The present subject matter can have the advantage that the therapy changes that take place every iteration eventually bring the population closer to the balance between the ranges 706, 708, and 710 that the optimal data 704 has. For example, at a time around week two in the data 702 (i.e., after one iteration of two weeks), the range 708 reaches a somewhat stable value (here, about 49%) and after that there are only small fluctuations that do not reflect an upward or downward trend. It can be said that the data 702 converges around week two in this example.

Moreover, the present subject matter can reach convergence relatively quickly. For example, after about one iteration (here, two weeks), convergence can be obtained. This can eliminate or reduce the occurrence of negative effects of the health and/or the quality of life of the population.

The determination of a TDBD mismatch can trigger adjustment of insulin therapy (e.g., one or more settings) and/or generation of a communication. One or more other values can also be determined based on the TDBD mismatch value. In some implementations, an insulin sensitivity factor (ISF) for the person with diabetes can be determined. For example, the ISF can be determined using a trivariate relationship. Trivariate lognormal distributions of basal rate (BR), carbohydrate-to-insulin ratio (CR), and ISF can be combined to produce a population. The population can be visually represented in a graph with ISF on a vertical axis, BR on one horizontal axis, and CR on another horizontal axis. The distribution of the population can resemble an ellipsoid. For example, the major axis of the ellipsoid can be described by the intersection of two planes, such as: CR=60.4*TDBD^{-0.573} ISF=521*TDBD⁻⁰⁷⁸⁹ or. As such, either ISF or CR, or both, can be determined or estimated using a trivariate relationship.

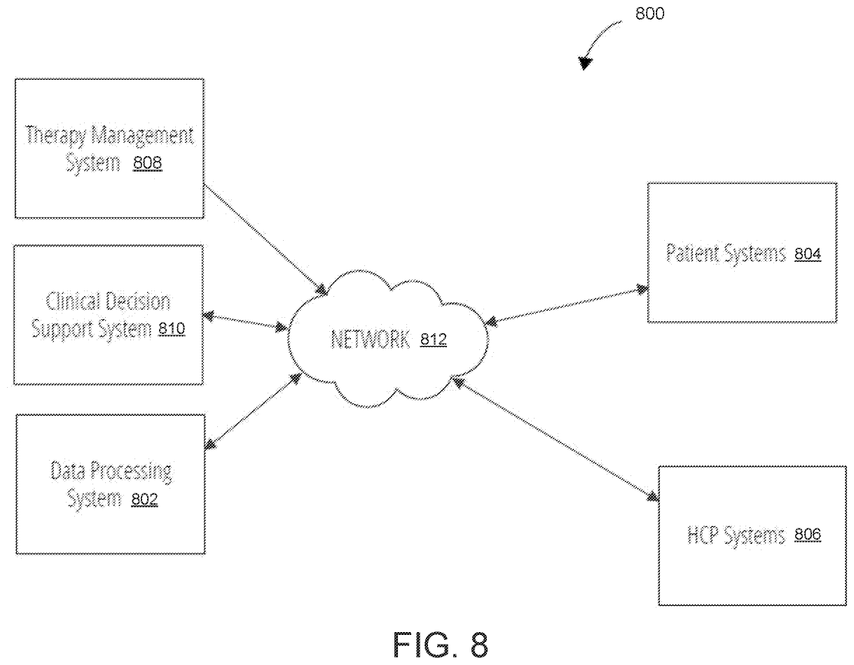

FIG. 8 shows a simplified block diagram of an example of a system for insulin-based management of diabetes. Any suitable technique used by one of ordinary skill in the art in the field of data science to calculate and/or express probabilities may be used with disclosed embodiments. Some embodiments relate, generally, to insulin therapy systems and elements thereof that incorporate systems, methods and devices for TDBD-mismatch determination. FIG. 8 shows a system 800 for insulin therapy, in accordance with disclosed embodiments. The system 800 can be used with one or more other examples described elsewhere herein.

In the embodiment shown in FIG. 8, data processing system 802, clinical decision support system 810, and therapy management system 808 are computing platforms configured, generally, to provide various services related to insulin therapy, in whole or in part, to each other and to HCP systems 806 and patient systems 804. The data processing system 802, clinical decision support system 810, therapy management system 808, HCP systems 806, and patient systems 804 can be connected by at least one network 812 (e.g., the Internet). HCP systems 806 may include, for example, portals, dashboards, electronic medical record systems, computing platforms executing the same, and more.

In some embodiments, therapy management system 808 may be one or more computing platforms configured to receive and store therapy data (such as therapy data 212) and physiological parameters about patients, issues alarms and alerts, and manages therapy settings for insulin delivery systems—all related to insulin-based management of a PWD's diabetes.

In some embodiments, clinical decision support system 810 may be one or more computing platform configured as a health data technology system for assisting HCPs with clinical decision making tasks, and more specifically in this example, assist HCPs with clinical decision making tasks related to a PWDs insulin therapy. In disclosed embodiments, clinical decision support system 810 is configured to assist with insulin-based management of diabetes, and automatically analyzes therapy data 212, identifies clinically relevant patterns in a PWD's therapy from therapy data 212, and provides data and recommendations to HCP systems 806 based on those patterns. A goal of embodiments of clinical decision support system 810 is to improve outcomes for PWDs by facilitating communication of clinically relevant “insights” about a PWD's insulin-based therapy to patient systems 804 and HCP systems 806 as well as by facilitating communication of therapy related advice from HCP systems 806 to patient systems 804.

In disclosed embodiments, data processing system 802 may be one or more computing platforms configured to process therapy data 212 stored at, or received from, therapy management systems 808 and/or clinical decision support system 810. In one embodiment, data processing system 802 may, among other things, include one or more elements of computing platform 200, including trained TDBD regressor 204. In this manner, data processing system 802 may be configured to perform TDBD mismatch determination for therapy management system 808 and/or clinical decision support system 810.

By way of example, data processing system 802 may perform TDBD-mismatch determination on therapy data 212 stored at clinical decision support system 810 and provide one or more reports 220 detailing one or more labeled retrospective time periods, as well as one or more determinations regarding TDBD mismatch. Clinical decision support system 810 may use the data in reports 220 to trigger insights and/or recommendations that it sends to HCP systems 806. Upon HCP system 806 accessing messages from clinical decision support system 810, data from reports 220 may be included in such message or accessible by HCP systems 806—e.g., accessible if HCP systems 806 requests data to support an insight or recommendation described in a message.

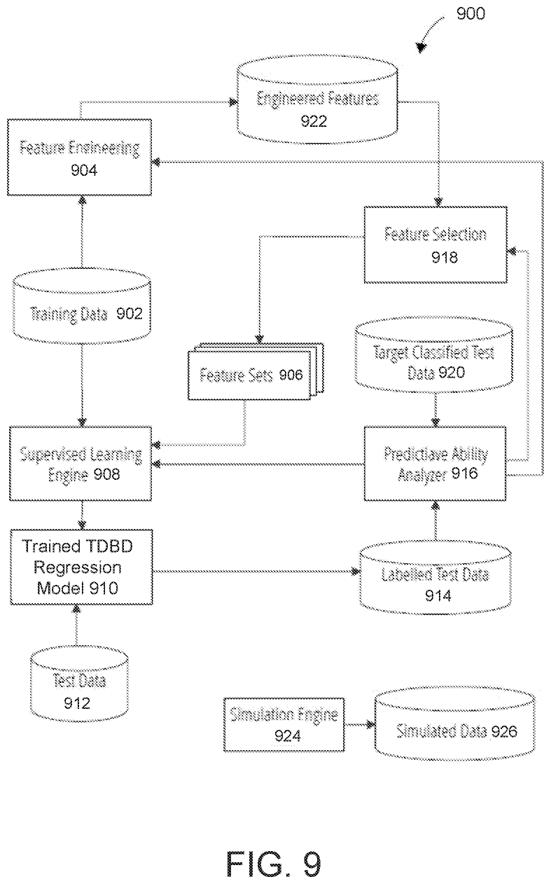

FIG. 9 shows a functional block diagram of system 900 for training a TDBD regression model (such as trained TDBD regressor 204) using machine learning techniques, in accordance with disclosed embodiments. The system 900 can be used with one or more other examples described elsewhere herein.

In a contemplated operation, supervised learning engine 908 trains trained TDBD regression model 910 using training data 902 and sets of engineered features (i.e., feature sets 906) selected for model training purposes. In some embodiments, trained TDBD regression model 910 is a function or algorithm that determines a TDBD mismatch. An initial “best guess” may be used for trained TDBD regression model 910 which is then continually improved by supervised learning engine 908. In disclosed embodiments, trained TDBD regression model 910 and supervised learning engine 908 may implement any suitable supervised learning algorithms and ensemble methods thereof for performing embodiments of the disclosure, including, for example, a linear support vector regressor (SVR), an SVR with radial basis function (RBF) kernel, Lasso, or Ridge regression model. Disclosed embodiments may also implement supervised learning algorithm(s) that do not use feature selection, including, for example, one class support vector machine (SVM) without feature selection, and logistic regression without feature selection.

In one embodiment, training data 902 is labeled therapy data associated with one or more PWDs. PWDs may be chosen so they are representative of a desired domain of PWD physiologies, eating behaviors, exercise behaviors, sleeping behaviors, diurnal profile variation, and more.

In disclosed embodiments, feature sets 906 are sub-sets of features engineered (i.e., formed) in the training data 902 and used by supervised learning engine 908 to train any classifier. In one embodiment, feature sets 906 are created using a feature selection process for selecting a subset of features included in a feature domain created using feature engineering techniques. Features in a feature domain may include, for example, one or more of the features identified in the table below:

TABLE I

| Feature | Description |
|---|---|
| tl54 | time below a blood glucose value of 54 |
| tl70 | time below a blood glucose value of 70 |
| tir | time within a blood glucose range |
| tg180 | time above a blood glucose value of 180 |
| tg250 | time above a blood glucose value of 250 |
| a1c | patient identifier |
| geoMean | a geometric mean of blood glucose values |
| geoStd | a geometric standard deviation of the blood |
| | glucose values |
| loss | a loss function regarding the blood glucose |
| | values |
| tdbd_programmed | a programmed TDBD value |
| cr_programmed | a programmed carbohydrate-to-insulin ratio |
| isf-programmed | a programmed insulin sensitivity factor |
| muStarOvernight | a geometric mean of blood glucose values |
| | overnight |
| sigmaStarOvernight | a geometric standard deviation of the blood |
| | glucose values overnight |
| muStarAfterMeal | a geometric mean of blood glucose values |
| | after a meal |
| sigmaStarAfterMeal | a geometric standard deviation of the blood |
| | glucose values after a meal |
| muStarAfterCorrection | a geometric standard deviation of the blood |
| | glucose values after a correction |

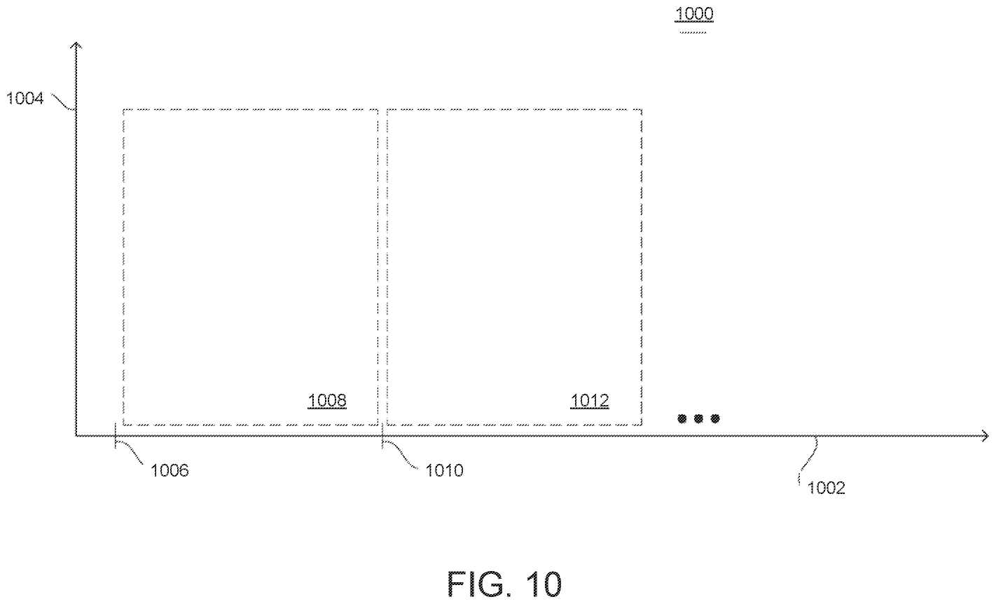

Some of the above feature sets (sometimes referred to as segments) relate to situations after a meal or overnight. In some implementations, the definitions of such feature sets can take into account insulin action profiles. For example, an insulin action profile of an insulin medication can be characterized by at least characteristics regarding the onset of the insulin therapeutic effect, the peak of the insulin therapeutic effect, or the duration of the insulin therapeutic effect. In some implementations, after-meal or overnight segments involve looking at parts of data where only a long-acting dose is active (e.g., more than six hours after the last rapid-action dose and before the next rapid-action dose), and at data showing the outcome of a rapid-acting dose (e.g., 2-6 hours post-meal for lows, 4-6 hours post-meal for a geometric mean). Examples relating to time ranges for data selection are described below with regard to FIG. 10.

That is, one or more feature sets can be constructed, such as by a selection process at a feature engineering stage. The feature selection process can be performed using the feature sets and the data to obtain a training feature set. Feature sets 906 may be selected from the feature domain using any suitable feature selection technique or combination of techniques for trying features in the feature domain and identifying important features, including, for example, sequential forward feature selection, sequential backward elimination, and tree-based feature selection algorithms.

Labeled test data 914 is test data 912 classified and labeled by a trained TDBD regression model 910 during successive iterations of system 900. The system 900 includes target classified test data 920. Labelled test data 914 is the “true” or “target” labels for test data 912. Stated another way, it is the labeling result that is the target for trained TDBD regression model 910. Predictive ability analyzer 916 assess the predictive ability of trained TDBD regression model 910 by comparing the labels of the regressed test data 914 that were predicted by the trained TDBD regression model 910 to the true values in the target test data. Any suitable technique for calculating and/or assessing validity of a model may be used by predictive ability analyzer 916, including for example, precision, recall, number of detected events versus number of true events, confusion matrix, area-under-the-free-curve (AUC), receiver operating characteristics (ROC) curve. Techniques such as Grid search combined with cross validation, and N-fold cross-validation can be used for hyper parameter tuning of a regressor.

Feature selection 918 receives assessment results from predictive ability analyzer 916 and, in response, changes feature sets 906 to attempt to improve accuracy and/or attempt to simplify feature sets 906. As non-limiting examples, changes to feature sets 906 may include, changing weighting for features of feature sets 906, adding features to feature sets 906 to attempt to improve accuracy of predictions, removing unnecessary features from feature sets 906, and combinations thereof.

Feature engineering 904 receives assessment results from predictive ability analyzer 916 and, in some cases, performs feature engineering techniques to extract new features from test data 912 and add those features to engineered features 922. These new features may be used in the feature selection process by feature selection 918.

In one embodiment, system 900 may include simulation engine 924 configured to generate simulation data 926 from which training data 902 and test data 912 may be obtained. Simulation engine 924 may be configured to simulate insulin therapy scenarios for a variety of PWDs. PWD profiles are created that represent a cross-section of PWDs in terms of characteristics such as physiology (e.g., age, weight, height, complicating health conditions, diurnal profile variation, etc.), lifestyle (e.g., eating behaviors, exercise behaviors, sleeping behaviors, etc.), socio-economic factors (e.g., income, race, geographic location, marriage status, child status, etc.), differences in how PWDs measure and track meal intake, and differences in the operation and quality of insulin delivery systems and components the PWDs use. In one embodiment, simulation engine 924 is configured to model for missing therapy data due to, for example, lost components, failure to input therapy related data, failure to wear a glucose monitor, and lost Bluetooth connection.

FIG. 10 shows an example of selecting time periods for evaluating blood glucose data (e.g., TDBD data, including, but not limited to, a TDBD mismatch). The following examples can be used in combination with, or can include aspects of, one or more other examples described elsewhere herein.

A diagram 1000 includes a horizontal axis 1002 relative to which time can be indicated, and a vertical axis 1004 relative to which a measure of insulin therapy successfulness can be indicated. In some implementations, the vertical axis 1004 indicates a measured or estimated level of blood glucose in a person with diabetes (e.g., measured in mg/dL).

A dosage time 1006 is here indicated on the horizontal axis 1002. In some implementations, the dosage time 1006 corresponds to the actual, estimated, or inferred point in time when the person with diabetes ingested insulin. For example, the dosage time 1006 can correspond to a most recent ingestion of rapid-acting insulin (e.g., as indicated by one or more capping events of a pen cap, as determined by monitoring a cap sensor on the pen cap).

A data inclusion area 1008 is schematically indicated in the diagram 1000. In some implementations, the data inclusion area 1008 is positioned to begin at, in conjunction with, or based on, the dosage time 1006, and to extend for a predefined amount of time thereafter. For example, the data inclusion area 1008 can correspond to a certain number of hours immediately following ingestion of a dose of rapid-acting insulin. The data inclusion area 1008 indicates at least one type of data from a greater data trove, such as a collection of blood glucose data over a plurality of days, that may be identified for analysis. For example, when evaluating the success of a rapid-acting insulin therapy, data falling within the data inclusion area 1008 can be separated from the rest of the data and subjected to analysis (e.g., the training data 902 and/or the test data 912 in FIG. 9 can be selected or classified based on the data inclusion area 1008). For example, variability of blood glucose values can be determined for a selected period of time, and the data inclusion area 1008 can define how the period of time is selected.

A time 1010 is indicated on the horizontal axis 1002. In some implementations, the time 1010 corresponds to the actual, estimated, or inferred point in time when the person with diabetes is considered to begin fasting. For example, the time 1010 can correspond to a time following sufficiently long after a most recent ingestion of rapid-acting insulin that it can be assumed only long-acting insulin will be affecting the person with diabetes (until a next dose of rapid-acting insulin).

A data inclusion area 1012 is schematically indicated in the diagram 1000. In some implementations, the data inclusion area 1012 is positioned to begin at, in conjunction with, or based on, the time 1010, and to extend for a predefined amount of time thereafter. For example, the data inclusion area 1012 can correspond to a certain number of hours immediately following the time when the most recently ingested dose of rapid-acting insulin is deemed to no longer be active or active to any significant extent (e.g., this can be considered fasting time or sleeping time). That is, the data inclusion area 1012 can be considered as following the data inclusion area 1008. In other words, the period of time of the data inclusion area 1012 can be following the period of time of the data inclusion area 1008. Analysis can be performed on the data corresponding to the data inclusion area 1012 whether or not any data corresponding to the data inclusion area 1008 (or any other data inclusion area) is also being analyzed.

The data inclusion area 1012 indicates at least one type of data from a greater data trove, such as a collection of blood glucose data over a plurality of days, that may be identified for analysis. In some implementations, when evaluating the success of a long-acting insulin therapy, data falling within the data inclusion area 1012 can be separated from the rest of the data and subjected to analysis (e.g., the training data 902 and/or the test data 912 in FIG. 9 can be selected or classified based on the data inclusion area 1012). For example, variability of blood glucose values can be determined for a selected period of time, and the data inclusion area 1012 can define how the period of time is selected. More or fewer data inclusion areas than the data inclusion areas 1008 and 1012 can be used. In some implementations, the data inclusion area 1012 is defined based on the length of time between the dosage time 1006 and the time 1010, although the data inclusion area 1008—that is, the extent during which data relating to the dosage ingested at the dosage time 1006—may be shorter than such a length of time. That is, a type of insulin (e.g., rapid-acting) can be analyzed during a first period of time (e.g., corresponding to the data inclusion area 1008), and another type of insulin (e.g., long-acting) can be analyzed during a second period of time (e.g., corresponding to the data inclusion area 1012). In such a scenario, the second period of time can be selected as beginning after a third period of time (e.g., the length of time between the dosage time 1006 and the time 1010) following a dosage time of the first-mentioned type of insulin.

The present subject matter can be implemented in form of systems, apparatuses, and/or the performance of methods. In some implementations, a method includes receiving, in a computer system, data regarding insulin therapy treatment of a person with diabetes. For example, the therapy management system 808 can receive the data from the patient system 804 (FIG. 8). The data can relate to a time period and comprise blood glucose values for the person with diabetes and time-of-administration information for the insulin therapy treatment. For example, two weeks' worth of blood glucose values and time-of-bolus information corresponding to the first iteration (i.e., week zero) of the graph 500 (FIG. 5) can be received. The method can include determining, using the computer system and based on the blood glucose values and the time-of-administration information, a TDBD mismatch value for the person with diabetes. For example, the trained TDBD regression model 910 (FIG. 9) can be used for determining a TDBD mismatch value. The method can include generating, using the computer system, an output based on the determined TDBD mismatch value. In some implementations, the therapy management system 808 can generate an output to the patient system 804 and/or to the HCP system 806. For example, the output can include an instruction to adjust the insulin therapy setting(s) for the person in a specified way. As another example, the output can include a communication that does not necessarily include a numerical adjustment value for the insulin therapy setting (e.g., a recommendation to change insulin dosage or another parameter affecting the person).

In some implementations, a method includes obtaining, in a computer system, data regarding insulin-based management of diabetes. For example, the therapy management system 808 can receive the data from the patient system 804 (FIG. 8). The data can include blood glucose values and time-of-administration information for the insulin-based management of diabetes. For example, two weeks' worth of blood glucose values and time-of-bolus information corresponding to the first iteration (i.e., week zero) of the graph 500 (FIG. 5) can be received. The method can include training, using the computer system, a regression model using the data. For example, the TDBD regression model 122 (FIG. 1) can be trained. The regression model can be trained to determine a TDBD mismatch value. For example, the TDBD mismatch value can reflect a ratio between a programmed TDBD value and an unknown TDBD value for the person. The method can include obtaining, using the computer system, a TDBD mismatch value using the trained regression model.

Multiple candidate regression models can be trained and evaluated. In some implementations, any of the regression models mentioned herein can be trained on a common set of data (e.g., training dataset 106 in FIG. 1). A predictive ability can be determined for each of the multiple candidate regression models. In some implementations, both programmed TDBD mismatch and actual TDBD can be known in the training data (e.g., simulated data), and the difference between them, if any, can be evaluated. For example, a mean squared error can be determined for each of the multiple candidate regression models, where a relatively lower mean squared error corresponds to a relatively higher predictive ability of the candidate regression model. As such, the candidate regression model having the highest predictive ability can be identified.

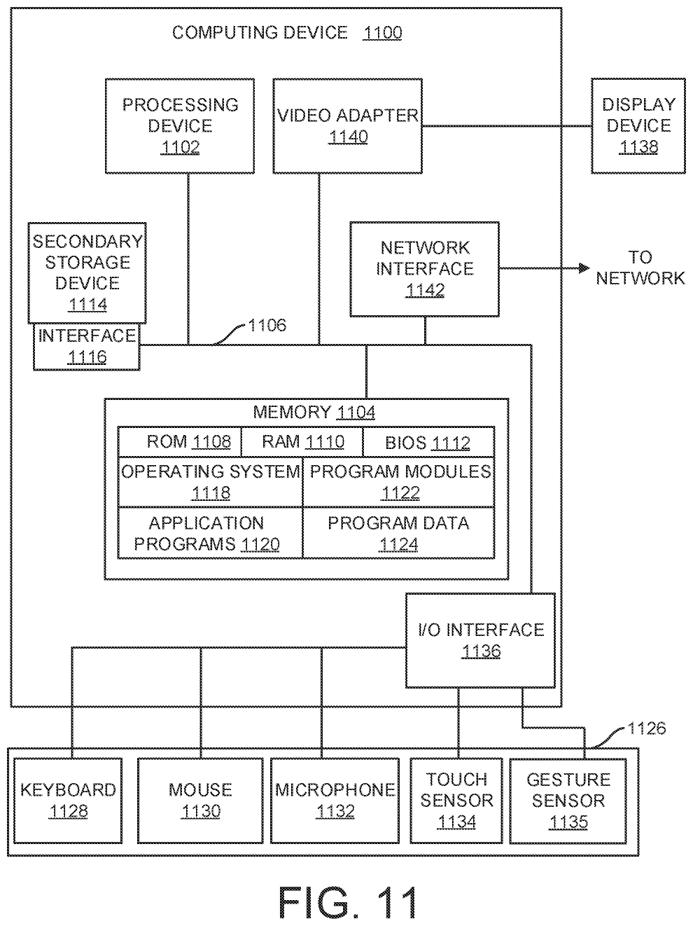

A system can include a non-transitory storage medium having stored therein data regarding insulin therapy treatment of a person with diabetes. For example, the therapy management system 808 can include the data received from the patient system 804 (FIG. 8). The data can relate to a time period and comprising blood glucose values for the person with diabetes and time-of-administration information for the insulin therapy treatment. For example, two weeks' worth of blood glucose values and time-of-bolus information corresponding to the first iteration (i.e., week zero) of the graph 500 (FIG. 5) can be received. The non-transitory storage medium can have stored therein a mismatch regression model. For example, the trained TDBD regression model 910 (FIG. 9) can be stored in the therapy management system 808. The system can include at least one processor (e.g., the processing device 1102 in FIG. 11). The processor can provide the data to the mismatch regression model and determine a TDBD mismatch value (e.g., an estimated ratio between a programmed TDBD value and an unknown actual TDBD value) for the person with diabetes. The system can include an output component (e.g., the display device 1138 and/or network interface 1142 in FIG. 11) to generate an output based on the determined TDBD mismatch value (e.g., an adjusted insulin therapy setting and/or a communication to the patient/physician).

FIG. 11 illustrates an example architecture of a computing device 1100 that can be used to implement aspects of the present disclosure, including any of the systems, apparatuses, and/or techniques described herein, or any other systems, apparatuses, and/or techniques that may be utilized in the various possible embodiments.

The computing device illustrated in FIG. 11 can be used to execute the operating system, application programs, and/or software modules (including the software engines) described herein.

The computing device 1100 includes, in some embodiments, at least one processing device 1102 (e.g., a processor), such as a central processing unit (CPU). A variety of processing devices are available from a variety of manufacturers, for example, Intel or Advanced Micro Devices. In this example, the computing device 1100 also includes a system memory 1104, and a system bus 1106 that couples various system components including the system memory 1104 to the processing device 1102. The system bus 1106 is one of any number of types of bus structures that can be used, including, but not limited to, a memory bus, or memory controller; a peripheral bus; and a local bus using any of a variety of bus architectures.

Examples of computing devices that can be implemented using the computing device 1100 include a desktop computer, a laptop computer, a tablet computer, a mobile computing device (such as a smart phone, a touchpad mobile digital device, or other mobile devices), or other devices configured to process digital instructions.

The system memory 1104 includes read only memory 1108 and random access memory 1110. A basic input/output system 1112 containing the basic routines that act to transfer information within computing device 1100, such as during start up, can be stored in the read only memory 1108.

The computing device 1100 also includes a secondary storage device 1114 in some embodiments, such as a hard disk drive, for storing digital data. The secondary storage device 1114 is connected to the system bus 1106 by a secondary storage interface 1116. The secondary storage device 1114 and its associated computer readable media provide nonvolatile and non-transitory storage of computer readable instructions (including application programs and program modules), data structures, and other data for the computing device 1100.

Although the example environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. For example, a computer program product can be tangibly embodied in a non-transitory storage medium. Additionally, such computer readable storage media can include local storage or cloud-based storage.

A number of program modules can be stored in secondary storage device 1114 and/or system memory 1104, including an operating system 1118, one or more application programs 1120, other program modules 1122 (such as the software engines described herein), and program data 1124. The computing device 1100 can utilize any suitable operating system, such as Microsoft Windows™, Google Chrome™ OS, Apple OS, Unix, or Linux and variants and any other operating system suitable for a computing device. Other examples can include Microsoft, Google, or Apple operating systems, or any other suitable operating system used in tablet computing devices.

In some embodiments, a user provides inputs to the computing device 1100 through one or more input devices 1126. Examples of input devices 1126 include a keyboard 1128, mouse 1130, microphone 1132 (e.g., for voice and/or other audio input), touch sensor 1134 (such as a touchpad or touch sensitive display), and gesture sensor 1135 (e.g., for gestural input. In some implementations, the input device(s) 1126 provide detection based on presence, proximity, and/or motion. In some implementations, a user may walk into their home, and this may trigger an input into a processing device. For example, the input device(s) 1126 may then facilitate an automated experience for the user. Other embodiments include other input devices 1126. The input devices can be connected to the processing device 1102 through an input/output interface 1136 that is coupled to the system bus 1106. These input devices 1126 can be connected by any number of input/output interfaces, such as a parallel port, serial port, game port, or a universal serial bus. Wireless communication between input devices 1126 and the input/output interface 1136 is possible as well, and includes infrared, BLUETOOTH® wireless technology, 802.11a/b/g/n, cellular, ultra-wideband (UWB), ZigBee, or other radio frequency communication systems in some possible embodiments, to name just a few examples.

In this example embodiment, a display device 1138, such as a monitor, liquid crystal display device, projector, or touch sensitive display device, is also connected to the system bus 1106 via an interface, such as a video adapter 1140. In addition to the display device 1138, the computing device 1100 can include various other peripheral devices (not shown), such as speakers or a printer.

The computing device 1100 can be connected to one or more networks through a network interface 1142. The network interface 1142 can provide for wired and/or wireless communication. In some implementations, the network interface 1142 can include one or more antennas for transmitting and/or receiving wireless signals. When used in a local area networking environment or a wide area networking environment (such as the Internet), the network interface 1142 can include an Ethernet interface. Other possible embodiments use other communication devices. For example, some embodiments of the computing device 1100 include a modem for communicating across the network.

The computing device 1100 can include at least some form of computer readable media. Computer readable media includes any available media that can be accessed by the computing device 1100. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 1100.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 11 is also an example of programmable electronics, which may include one or more such computing devices, and when multiple computing devices are included, such computing devices can be coupled together with a suitable data communication network so as to collectively perform the various functions, methods, or operations disclosed herein.

The terms “substantially” and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. Also, when used herein, an indefinite article such as “a” or “an” means “at least one.”

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

A number of implementations have been described. Nevertheless, it will be understood that various modifications may be made without departing from the spirit and scope of the specification.

In addition, the logic flows depicted in the figures do not require the particular order shown, or sequential order, to achieve desirable results. In addition, other processes may be provided, or processes may be eliminated, from the described flows, and other components may be added to, or removed from, the described systems. Accordingly, other implementations are within the scope of the following claims.

While certain features of the described implementations have been illustrated as described herein, many modifications, substitutions, changes and equivalents will now occur to those skilled in the art. It is, therefore, to be understood that appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

What is claimed is:

1. A method comprising:
receiving, in a computer system, data regarding insulin therapy treatment of a person with diabetes, the data relating to a time period and comprising blood glucose values for the person with diabetes and time-of-administration information for the insulin therapy treatment;
determining, using the computer system and based on the blood glucose values and the time-of-administration information, a total daily basal dose (TDBD) mismatch value for the person with diabetes; and
generating, using the computer system, an output based on the determined TDBD mismatch value.

2. The method of claim 1, wherein determining the TDBD mismatch value comprises using a regression model.

3. The method of claim 2, further comprising training the regression model before determining the TDBD mismatch value, the regression model trained using simulated insulin therapy treatment data.

4. The method of claim 1, wherein generating the output comprises adjusting an insulin therapy setting for the person with diabetes using the TDBD mismatch value.

5. The method of claim 4, wherein the insulin therapy setting is adjusted by an entirety of the TDBD mismatch value.

6. The method of claim 4, wherein the insulin therapy setting is adjusted by a portion of the TDBD mismatch value.

7. The method of claim 4, further comprising communicating the adjusted insulin therapy setting using the computer system.

8. The method of claim 7, wherein the adjusted insulin therapy setting is communicated to at least one of the person with diabetes or a physician for the person with diabetes.

9. The method of claim 1, wherein generating the output comprises generating a communication.

10. The method of claim 9, wherein the communication is generated to at least one of the person with diabetes or a physician for the person with diabetes.

11. The method of claim 1, further comprising determining, using the computer system and based on the TDBD mismatch value, an insulin sensitivity factor (ISF) for the person with diabetes.

12. The method of claim 11, wherein the ISF is determined using a trivariate relationship.

13. The method of claim 11, wherein the ISF is determined based on a new insulin therapy value calculated using the TDBD mismatch value.

14. The method of claim 1, further comprising determining, using the computer system and based on the TDBD mismatch value, a carbohydrate-to-insulin ratio (CR) for the person with diabetes.

15. The method of claim 14, wherein the CR is determined using a trivariate relationship.

16. The method of claim 14, wherein the CR is determined based on a new insulin therapy value calculated using the TDBD mismatch value.

17. The method of claim 1, wherein a programmed TDBD value for the person with diabetes for the time period is known, and wherein generating the output comprises adjusting the programmed TDBD value using the TDBD mismatch value.

18. The method of claim 17, wherein adjusting the programmed TDBD value comprises determining a new TDBD value for the person with diabetes by dividing the programmed TDBD value with the TDBD mismatch value.

19. The method of claim 1, wherein the time period is two weeks.

20. A method comprising:
obtaining, in a computer system, data regarding insulin-based management of diabetes, the data including blood glucose values and time-of-administration information for the insulin-based management of diabetes;
training, using the computer system, a regression model using the data, the regression model trained to determine a total daily basal dose (TDBD) mismatch value; and
obtaining, using the computer system, a TDBD mismatch value using the trained regression model.

21. The method of claim 20, further comprising:
training multiple candidate regression models using the data; and
selecting one of the multiple candidate regression models to be the regression model, the selection comprising:
determining a predictive ability for each of the multiple candidate regression models; and
identifying one of the multiple candidate regression models having a highest predictive ability of the determined predictive abilities.

22. The method of claim 20, further comprising:
constructing feature sets, wherein each of the feature sets is constructed by selecting one or more features to include in the feature set; and
performing a feature selection process using the feature sets and the data to obtain a training feature set.

23. The method of claim 22, wherein the training feature set includes at least some of:
a feature reflecting time below a first blood glucose value;
a feature reflecting time below a second blood glucose value;
a feature reflecting time with blood glucose within a predefined range;
a feature reflecting time above a third blood glucose value;
a feature reflecting time above a fourth blood glucose value;
a feature reflecting a geometric mean of blood glucose values;
a feature reflecting a geometric standard deviation of the blood glucose values;
a feature reflecting a loss function regarding the blood glucose values;
a feature reflecting a programmed TDBD value;
a feature reflecting a programmed carbohydrate-to-insulin ratio; or
a feature reflecting a programmed insulin sensitivity factor.

24. The method of claim 20, further comprising:
simulating insulin-based management of diabetes; and
obtaining the data regarding insulin-based management of diabetes from simulating the insulin-based management of diabetes.

25. A system comprising:
a non-transitory storage medium having stored therein:
data regarding insulin therapy treatment of a person with diabetes, the data relating to a time period and comprising blood glucose values for the person with diabetes and time-of-administration information for the insulin therapy treatment; and
a mismatch regression model; and
at least one processor to provide the data to the mismatch regression model and determine a total daily basal dose (TDBD) mismatch value for the person with diabetes.

26. The system of claim 25, further comprising an output component to generate an output based on the determined TDBD mismatch value.

27. The system of claim 26, wherein the output comprises an adjustment of an insulin therapy setting for the person with diabetes using the TDBD mismatch value.

28. The system of claim 26, wherein the output comprises a communication.
